(19) United States
(12) Patent Application Publication
(10) Pub. No.: US 2013/0204117 A1
(43) Pub. Date: Aug. 8, 2013

(54) METHOD TO EVALUATE RAW MEDICAL DATA

(71) Applicants: Christina Bauer, Buckenhof (DE); Thomas Blum, Neunkirchen A. Br (DE)

(72) Inventors: Christina Bauer, Buckenhof (DE); Thomas Blum, Neunkirchen A. Br (DE)

(21) Appl. No.: 13/760,455

(22) Filed: Feb. 6, 2013

(30) Foreign Application Priority Data
Feb. 6, 2012 (DE) 102012201708.9

(51) Int. Cl. A61B 5/055; A61B 5/00

(52) U.S. Cl. CPC A61B 5/055; A61B 5/7225; A61B 5/0013
USPC 600/410

(57) ABSTRACT

In a method to evaluate raw data acquired with a medical image acquisition device, such as raw three-dimensional magnetic resonance spectroscopy data, wherein at least one evaluation algorithm is applied to the raw data to determine output data, the raw data are transferred from the medical acquisition device to at least one external service having a different operator from the image acquisition device. The evaluation algorithm is applied to the raw data at the external service and the output data are transferred back from the external service to the medical acquisition device.

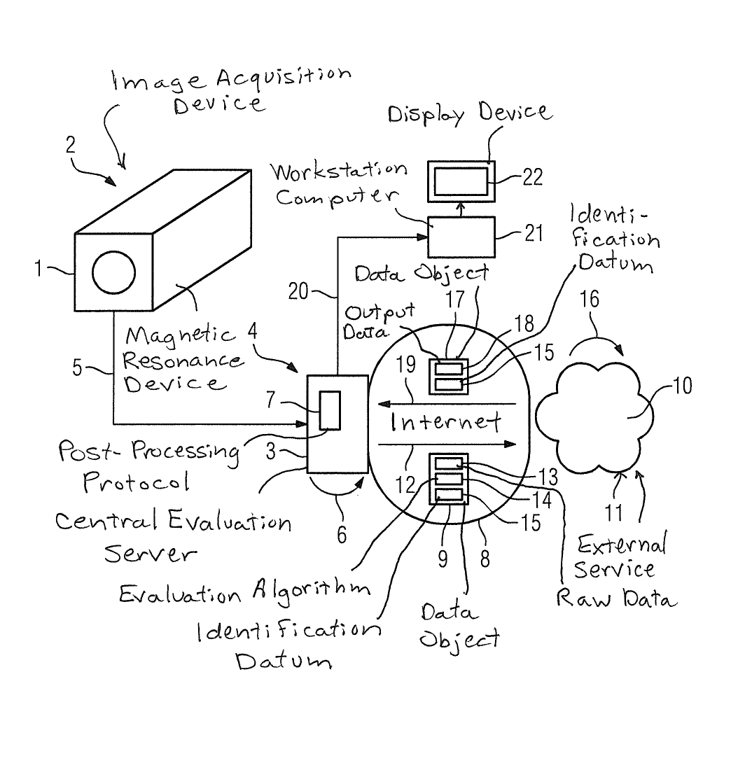

### BACKGROUND OF THE INVENTION

1. Field of the Invention

The invention concerns a method to evaluate raw data acquired with a medical image acquisition device, in particular three-dimensional raw magnetic resonance spectroscopy data, of the type wherein at least one evaluation algorithm is applied to the raw data to determine output data.

2. Description of the Prior Art

In the operation of image acquisition devices in modern medical, large sets of raw data (for example image data or other measurement data) frequently accumulate. The raw data are then evaluated by algorithms that are complicated (which algorithms can be realized via software and/or electronics) in order to obtain the desired information, in particular information forming the basis of a diagnosis. Because these evaluation algorithms must deal with a large amount of raw data and frequently also themselves represent complex post-processing steps, increased computing power is required at least intermittently, which can slow the typical operation by the operator of the image acquisition device (a hospital, for example).

One example of such a time-consuming evaluation process is magnetic resonance spectroscopy. This is a clinical, non-invasive method with which the concentration of specific metabolic products (metabolites) in human tissue can be determined, by raw data being acquired with a magnetic resonance device as an image acquisition device. The concentration of specific substances such as N-acetyl aspartate, choline and creatine in healthy tissue are known. Variations of the concentrations indicate specific illnesses.

A particular field of application of magnetic resonance spectroscopy is the detection of tumors of the prostate. For this purpose, three-dimensional, raw magnetic resonance spectroscopy data are acquired with a method called “chemical shift imaging” (CSI), which is why such raw data are frequently also designated as 3D-CSI data. Such raw data are post-processed using computer-intensive post-processing steps employing evaluation algorithms in order to obtain clinically interpretable information. The computing time is in the range of a minute, for example, even for modern, well-equipped computers.

For example, it is known to use a client-server architecture with a central evaluation server, wherein the evaluation algorithms for spectroscopy post-processing are realized (implemented) at the evaluation server. It has been proposed to implement the calculations within the scope of the evaluation algorithms in multiple parallel threads at the evaluation server in order to further minimize the calculation time. Even given powerful servers, however, this does not lead to acceptable run times of the algorithms, and, moreover, the procurement of even more powerful computers or computer farms is not efficient, and thus not cost-effective, because these would be under-utilized due to the infrequent occurrence of such calculations.

### SUMMARY OF THE INVENTION

An object of the invention is to provide a method with which the evaluation time of complex evaluation algorithms that act on raw medical data can be reduced.

To achieve this object in accordance with the invention, in a method of the aforementioned type the raw data are transferred to at least one external service having a different operator from the image acquisition device; and the evaluation algorithm is applied to the raw data at the external service, and the output data are transferred back to the image acquisition device from the external service.

The basis of the present invention is thus to achieve an improved, faster evaluation of the raw data through the use of what is known as “cloud computing”. Cloud computing designates services that provide computing power upon request, while the user of this computing power does not personally possess, operate or organize the devices required for this. Such calculation resources can equally be hardware (such as networks, servers, storage devices) or software (such as services and applications).

Cloud computing (in which external resources are used that the operator of the image acquisition device does not personally need to operate) represents a reasonable alternative with regard to medical evaluation procedures since, and via communication connections with a number of image acquisition devices, agreements with various operators of image acquisition devices, or even use beyond the evaluation of raw data, the corresponding devices can achieve a utilization that is cost-effective. The operator of the image acquisition device need no longer be concerned about or responsible for the organization and the operation of devices that allow the evaluation of the raw data within a desired and predetermined time window; rather, a request for the sufficient computing power is sent to the operator of the cloud (thus the service), so this operator is ultimately responsible for always providing the necessary computing power in whatever manner, and thus enabling a faster evaluation of the raw data into output data. The term “cloud” means that it is ultimately irrelevant in what manner or with which devices and which software the required computing power is provided; rather, this is left entirely to the operator of the cloud.

Overall, the time-consuming evaluation of the raw data via the evaluation algorithms is thus no longer implemented at an evaluation server or another computer of the operator of the image acquisition device, for example, but rather is relocated externally so that the post-processing time until clinically interpretable information is present at the image acquisition device can be markedly reduced. This reduction of the post-processing time is an improvement that directly affects the clinical acceptance of the product and the examination method that require such intensive computing power.

For example, an agreement can be made with a cloud provider and the cloud infrastructure can be used. For example, the computationally intensive post-processing is implemented in a computing center of the cloud provider (thus the operator of the service). The evaluated output data that are thus obtained are transmitted back to the sending computer of the operator of the image acquisition device and there are possibly subjected to additional, less computationally intensive evaluation steps and/or are displayed at a viewing station (for example a workstation computer).

In a further embodiment of the present invention, the processing of the raw data and the output data takes place at least in part at a computer (in particular a server) of the operator of the image acquisition device, and the raw data are transferred from this computer to the service and the output data are transferred from the service to the computer. Initially, the data are thus acquired with the image acquisition device as is conventional, and a typical evaluation task flow (for example a spectroscopy task flow) is started, for example, and an evaluation server automatically selects a post-processing protocol, for example. If the processing of the data via the at least one evaluation algorithm is next in line, the raw data are transferred to the service (thus the cloud). There the data are evaluated by means of the evaluation algorithm, and the output data arising as a result are sent back to the computer of the operator of the image acquisition device (thus in particular to the evaluation server), which computer possibly proceeds with the post-processing program until diagnostically interpretable information are generated that can be displayed to a physician, for example.

In an embodiment of the present invention, in the case of raw data associated with a patient, the raw data are transferred in anonymous form to the service. In order to increase data security, only the data necessary for calculation within the scope of the evaluation algorithm are thus transferred to the service. No patient data (for example the name of the patient, the address of the patient and the like) belong among these. Even if data thus arrive at the wrong destination or the like, they are a pure collection of raw data (and possibly anonymous additional parameters) that are not interpretable, such that overall the security of the procedure is increased.

However, additionally or alternatively the raw data and/or the output data are transmitted via a secure connection and/or in encrypted form. Although cases exist in which—given an anonymization of the raw data (i.e. a generation thereof in anonymous form)—this is already sufficient to guarantee the sufficient data security, an additional protection can be achieved—in particular when additional information subject to data protection are transferred—in that a secure connection to the service is used. For example, a VPN tunnel can be used as a secure connection, but other secure transfer possibilities as are known in the prior art are naturally also applicable. An encryption of the data with fundamentally known encryption algorithms is also conceivable to further increase the security.

At this point it is noted that professional cloud solutions already provide for the security in the service itself, which means that security mechanisms (firewalls, for example) are already implemented in the service so that the data security in the cloud is ensured.

The raw data and/or the output data can be transferred via the Internet. In this case, no dedicated, private communication connection from a computer of the operator of the image acquisition device is required for the service; rather, the Internet (which is present anyway and widely utilized today) is used, which provides the suitable communication connections for transfer of the raw data and the output data.

An identification datum (in particular a unique DICOM ID) can be transferred with the raw data and the output data, which identification datum is used for association with the transferred (in particular anonymized) data. In particular when multiple raw data sets are transferred from one location, it is relevant that the output data that are received back are correctly associated, even when the raw data have in principle been transferred in an anonymized manner. In these cases, a unique identification datum can consequently be transferred with the raw data and the output data, wherein the unique DICOM ID in particular suggests itself here because the raw data for the most part exist anyway in the DICOM format. Naturally, it is also conceivable to generate a suitable ID at the transmission source.

Various models of cloud computing (thus services that can be used within the scope of the present invention) are known that are widespread to different extends. It is basically conceivable that a cloud of the “Software as a Service” (SaaS) concept is used as a service so that the required evaluation algorithms are already present at the service, and ultimately only the raw data are still required. However, this requires a stronger integration of the operator of the service in the overall development, in particular with regard to the evaluation algorithm, such that according to the invention a cloud solution of the “Platform as a Service” (PaaS) type is preferred.

The evaluation algorithm can be transferred to the service together with the raw data for use at the service. If a PaaS cloud is used as a service, this provides an infrastructure at which the customer (the operator of the image acquisition device) can realize his own applications. One example of such a service is “Windows Azure” from Microsoft. Because the evaluation algorithm is typically very small in comparison to the amount of raw data, a complete “data object” in which the evaluation algorithm is also already included can be sent to the service without any problems. As described, the runtime environment or, respectively, operating environment that is required for the evaluation algorithm is provided by the operator of the service. The service thus provides a defined operating environment to which the evaluation algorithm is adapted. It is therefore possible to develop evaluation algorithms specifically with regard to the service and then to include them correspondingly.

As mentioned, magnetic resonance spectroscopy is a medical application case in which the computing time represents an especially critical problem. The method according to the invention can consequently be applied particularly advantageously when raw data acquired with chemical shift imaging are in particular evaluated [sic] into output data indicating the concentration of at least one substance (in particular a metabolite). Here marked savings in the evaluation time can be achieved due to the use of the service.

### BRIEF DESCRIPTION OF THE DRAWING

The single FIGURE shows a system for implementation of the method according to the invention.

### DESCRIPTION OF THE PREFERRED EMBODIMENTS

An exemplary embodiment of the method according to the invention is presented in detail here with regard to the evaluation of raw magnetic resonance spectroscopy data, in particular raw 3D-CSI data. The raw data are accordingly initially acquired with a magnetic resonance device 1 as an image acquisition device 2. From there the raw data are transferred to a central evaluation server 3 as a computer 4 of the operator of the image acquisition device (Step 5). In Step 6, a post-processing protocol 7 that is suitable for the raw data is selected and executed automatically by the evaluation server 3. The method according to the invention applies at the point in time at which the raw data should be processed with an evaluation algorithm to determine output data.

Via a secure connection (here a VPN tunnel) over the Internet 8, a data object 9 is transferred to a service 11 realized via a cloud 10 (Step 12). The service 11 is a cloud 10 according to the concept of the “Platform as a Service”, which means the service 11 provides a clearly defined operating environment. In addition to the raw data 13, the evaluation algorithm 14 developed for this operating environment and a unique identification datum 15 (here the unique DICOM ID) are consequently also transferred in the data object 9. The evaluation algorithm 14 can be used directly by the service 11 in order to evaluate the raw data 13 into output data 18 because the suitable operating environment is provided.

In Step 12, the data object 9 includes no information whatsoever about the patient with which the raw data 13 are associated, which means that the data are transferred in an anonymized form. It is thereupon noted that an encryption of the data of the data object 9 can also additionally or alternatively take place.

As mentioned, the raw data 13 are now evaluated by the service 11 by means of the evaluation algorithm 14 (Step 16). This occurs in a markedly faster manner than could be realized at the evaluation server 3. As a result, output data are obtained that are transferred back to the evaluation server 3 (likewise via the Internet 8) as an additional data object 17 including the output data 18 and the identification datum 15 (Step 19). A VPN tunnel can also be used for this.

If additional steps of the post-processing protocol 7 are still to be provided, this now occurs at the evaluation server 3 (as is typical) before the data are transferred to a workstation computer 21 (Step 20) where they can be presented at a corresponding display device 22, for example to create a finding.

The method according to the invention is naturally also applicable to multiple evaluation algorithms. It is thus possible for multiple raw data to be transferred to the service 11 in various stages of evaluation in order to determine output data.

Precisely how the required computing power and the required operating environment are provided on the part of the cloud 10 is ultimately not relevant since the service 11 is provided by another operator than the image acquisition device 2 and the evaluation server 3. It is thus an external service that is not subject to the organization and supervision of the image acquisition device 2.

In the present exemplary embodiment, the concentrations of specific metabolites are ultimately provided as output data, or at least as information derived from these.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to evaluate data, comprising:
acquiring medical raw data with a medical image acquisition device operated by a medical device user at a medical device site, the acquired medical data being present in a computerized processor of said medical image acquisition device;
transferring said medical raw data from said device processor to at least one external surface, that is external of said medical device site and that is not operated by said device user;
at said external service, applying a computerized evaluation algorithm to said medical raw data, and thereby obtaining evaluated medical data; and
transferring the evaluated medical data back to said device processor at said medical device site from said external service.

2. A method as claimed in claim 1 comprising acquiring said medical raw data by operating a magnetic resonance apparatus, as said medical image acquisition device, to acquire raw three-dimensional magnetic resonance spectroscopy data, as said medical raw data.

3. A method as claimed in claim 1 comprising processing said medical raw data and said output data in said device processor.

4. A method as claimed in claim 1 wherein said medical raw data are associated with a patient, and transferring said medical raw data from said device processor to said external service in a form in which said patient is anonymous.

5. A method as claimed in claim 1 comprising transferring at least one of said medical raw data and said output data via a secure connection between said device processor and said external service.

6. A method as claimed in claim 5 comprising transferring said at least one of said medical raw data and said output data via a VPN tunnel as said secure connection.

7. A method as claimed in claim 1 comprising transferring at least one of said medical raw data and of said output data in encrypted form.

8. A method as claimed in claim 1 comprising transferring at least one of said medical raw data and of said output data via the Internet.

9. A method as claimed in claim 1 comprising transferring an identification datum with said medical raw data and with said output data that associates the medical raw data and the output data with a patient in anonymized form.

10. A method as claimed in claim 9 comprising using a unique DICOM identifier as said identification datum.

11. A method as claimed in claim 1 comprising transferring said evaluation algorithm together with said medical raw data to said external service from said device processor.

12. A method as claimed in claim 11 wherein external service applies said evaluation algorithm to the transferred medical raw data in a defined operating environment, and adapting said evaluation algorithm to said defined operating environment.

13. A method as claimed in claim 1 comprising acquiring said raw medical data with a magnetic resonance apparatus as said medical image acquisition device, and operating said magnetic resonance apparatus with a chemical shift imaging sequence to acquire said medical raw data, and evaluating said medical raw data at said external service by applying an evaluation algorithm to said medical raw data that identifies a concentration of at least one substance in a subject from which the medical raw data were acquired.

14. A method as claimed in claim 1 wherein said device processor has a computing power associated therewith, and comprising, at said external service, applying an evaluation algorithm to the transferred medical raw data that requires computing power that exceeds said computing power of said device processor.
